(19) United States
(12) Patent Application Publication
(10) Pub. No.: US 2010/0168149 A1
(43) Pub. Date: Jul. 1, 2010

(54) Camptothecin Analogue Compounds, a Process for Their Preparation and Pharmaceutical Compositions Containing Them

(76) Inventors: Gilbert Lavielle, La Celle Saint Cloud (FR); Patrick Hautefaye, Servon Brie Comte Robert (FR); Alain Pierre, Les Alluets Le Roi (FR); John Hickman, Paris (FR); Stephane Leonce, Versailles (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 11/990,065

(22) PCT Filed: Aug. 4, 2006
(86) PCT No.: PCT/FR2006/001901

§ 371 Date: Feb. 5, 2008

(30) Foreign Application Priority Data
Aug. 5, 2005 (FR) 05.08364

(51) Int. Cl. A61K 31/475; C07D 471/14; A61P 35/00

(52) U.S. Cl. 514/283; 546/51

(57) ABSTRACT

Compound of formula (I):

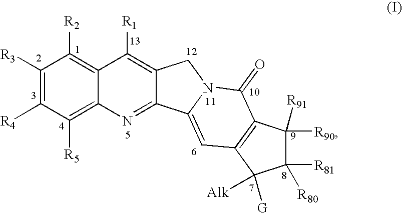

wherein:
R₁, R₂, R₃, R₄, R₅, R₈₀, R₉₀, R₈₁, R₉₁, Alk and G are as defined in the description.

Medicinal products containing the same which are useful in the treatment of cancer diseases.

The present invention relates to new aminoesterified camptothecin analogue compounds having a ketonic E ring, to a process for their preparation and to pharmaceutical compositions containing them.

Camptothecin (CPT), an alkaloid isolated from Camptotheca accuminata, is an anti-cancer agent having a broad spectrum of activity. Its insoluble nature has for a long time directed research towards the soluble salts of the compound, which have proved to be inactive and toxic.

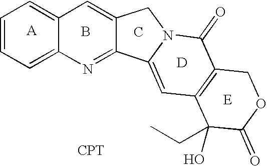

Another problem comes from the lack of stability of the E ring. In fact, in physiological media, the lactone function of the E ring is in equilibrium with the open hydroxy-acid form. The latter is inactive and seems to have a particular intrinsic toxicity [Cancer Research., 49, 1465 (1989); ibid, 49, 5077 (1989)]. Attempts at modifying this ring in order to make it more stable have been carried out; in particular, the cyclic oxygen atom has been replaced by a nitrogen or sulphur atom, but in each case there is loss of pharmacological activity, so confirming the importance of the lactone [Journal of Medicinal Chemistry, 32, 715 (1989)]. Other structural modifications of the E ring of CPT have been subsequently described, in particular in the patent specification EP 1 101 765. Those newer compounds are characterised by replacement of the lactone by a cyclic ketone function.

The present invention relates to camptothecin analogues having a ketone function on a five-membered E ring and at least one aromatic group bonded directly or indirectly to at least one of the carbon atoms selected from C₁, C₂, C₃, C₄ and C₁₃ of the quinoline moiety.

This modification provides the compounds of the invention with enhanced pharmacological activity, especially in respect of their cytotoxicity.

It will accordingly be possible to use them in the manufacture of medicaments for use in the treatment of cancer diseases.

The invention relates to compounds of formula (I):

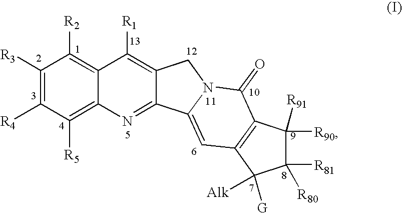

wherein:
Alk represents an alkyl group,
R₁, R₂, R₃, R₄ and R₅ are independently selected from a hydrogen atom, a halogen atom, an alkyl group, an alkenyl group, an alkynyl group, a polyhaloalkyl group, an optionally substituted cycloalkyl group, an optionally substituted cycloalkylalkyl group, a hydroxy group, a hydroxyalkyl group, an alkoxy group, an alkoxyalkyl group, a nitro group, a cyano group, an acyloxy group, a —C(O)—R group, and the groups —(CH₂)ₚ—NRₐR_{b}, —O—C(O)—NRₐR_{b} and —Z—Ar, wherein:
   R represents an alkyl group, an alkoxy group or an amino group (optionally substituted on the nitrogen atom by one or two alkyl groups),
   p is an integer from 0 to 6,
   Rₐ and R_{b} independently represent a hydrogen atom, an alkyl group, a cycloalkyl group, a cycloalkylalkyl group, an acyl group, an optionally substituted aryl group or an optionally substituted arylalkyl group, or Rₐ and R_{b} form together with the nitrogen atom carrying them a pyrrolyl, piperidyl or piperazinyl group, it being possible for each of those cyclic groups to be optionally substituted,
   Z represents a bond, an oxygen atom, a sulphur atom or a group selected from —Z′—S(O)—, —S(O)ᵣ—Z′— and —Z′—(CR_{c}R_{d})_{q}—Z″—,
   Ar represents an optionally substituted aryl or an optionally substituted heteroaryl group,
   Z′ and Z″, which are the same or different, represent an oxygen atom, a sulphur atom, a group —NRₑ— or a bond,
   R_{c}, R_{d} and Rₑ, which are the same or different, represent a hydrogen atom or an alkyl group,
   q represents an integer from 1 to 6,
   and r represents an integer 1 or 2,
   or two adjacent groups from R₂, R₃, R₄ and R₅ form together with the carbon atoms carrying them a group -T-(CR_{g}Rₕ)ₜ-T′-, wherein T and T′, which are the same or different, represent an oxygen atom, a sulphur atom or a group N—Rᵢ; R_{g} and Rₕ, which are the same or different, represent a hydrogen atom or a halogen atom; t is an integer from 1 to 3 inclusive; and Rᵢ represents a hydrogen atom, an alkyl group or a benzyl group,
R₈₀ and R₉₀ independently represent a hydrogen atom, a hydroxy group, an alkyl group or an alkoxy group,
R₈₁ and R₉₁ independently represent a hydrogen atom, an alkyl group, an alkenyl group or an alkynyl group, or, taken in pairs on adjacent carbon atoms, together form a bond or an oxirane group, or two geminal groups (R₈₀ and R₈₁) and/or (R₉₀ and R₉₁) together form an oxo group or a group —O—(CH₂)ₜ₁—O—, t₁ being an integer from 1 to 3 inclusive,
G represents a group *—XH or *—X—C(X′)-Alk′-G′, wherein:
   * represents the point of attachment to the C₇ carbon atom,
   X and X′, which are the same or different, represent an oxygen atom, a sulphur atom, an amino group or an alkylamino group,
   Alk′ represents an alkylene, alkenylene or alkynylene chain,
   G′ represents a hydrogen atom or a group NR₆R₇ wherein:
i) either R₆ and R₇ represent, each independently of the other, a hydrogen atom, an alkyl group, a cycloalkyl group, an optionally substituted aryl group, an optionally substituted arylalkyl group, an optionally substituted cycloalkyl group, an optionally substituted cycloalkylalkyl group, an optionally substituted heteroaryl group or an optionally substituted heteroarylalkyl group,
ii) or R₆ and R₇ form together with the nitrogen atom a 5- to 8-membered monocyclic heterocycloalkyl group

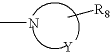

or a 5- to 11-membered bicyclic heterocycloalkyl group

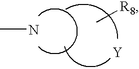

wherein:
Y represents a nitrogen atom, an oxygen atom or a CH₂ group and
R₈ represents a hydrogen atom, an alkyl group, an optionally substituted cycloalkyl group, an optionally substituted cycloalkylalkyl group, an optionally substituted aryl group, an optionally substituted arylalkyl group, an optionally substituted heterocycloalkyl group, an optionally substituted heterocycloalkylalkyl group, an optionally substituted heteroaryl group or an optionally substituted heteroarylalkyl group,
   to their enantiomers and diastereoisomers, and to addition salts thereof with a pharmaceutically acceptable acid or base,
   it being understood that at least one of the substituents R₁ to R₅ represents a group —Z—Ar,
   and it being understood that:
the term alkyl denotes a linear or branched chain of from 1 to 6 carbon atoms,
the term alkenyl denotes a linear or branched chain of from 2 to 6 carbon atoms containing from 1 to 3 double bonds,
the term alkynyl denotes a linear or branched chain of from 2 to 6 carbon atoms containing from 1 to 3 triple bonds,
the term alkylene denotes a linear or branched divalent radical containing from 1 to 6 carbon atoms,
the term alkenylene denotes a linear or branched divalent radical containing from 2 to 6 carbon atoms and from 1 to 3 double bonds,
the term alkynylene denotes a linear or branched divalent radical containing from 2 to 6 carbon atoms and from 1 to 3 triple bonds,
the term acyl denotes a linear or branched alkyl-carbonyl radical containing from 1 to 6 carbon atoms,
the term alkoxy denotes an alkyl-oxy radical, the alkyl group of which is linear or branched and contains from 1 to 6 carbon atoms,
the term acyloxy denotes an acyl-oxy radical, the acyl group of which is a linear or branched alkylcarbonyl radical,
the term aryloxyalkyl denotes an aryl-oxy-alkyl group, the alkyl group of which is linear or branched and contains from 1 to 6 carbon atoms,
the terms arylalkyl, cycloalkylalkyl, heteroarylalkyl and heterocycloalkylalkyl denote aryl-alkyl, cycloalkyl-alkyl, heteroaryl-alkyl and heterocycloalkyl-alkyl radicals, the alkyl groups of which denote a linear or branched chain of from 1 to 6 carbon atoms,
the term polyhaloalkyl denotes a linear or branched carbon chain containing from 1 to 3 carbon atoms and from 1 to 7 halogen atoms,
the term halogen denotes fluorine, chlorine, bromine or iodine atoms,
the term aryl denotes a phenyl, naphthyl, indanyl, indenyl, dihydronaphthyl or tetrahydronaphthyl group,
the term cycloalkyl denotes a monocyclic or bicyclic hydrocarbon group containing from 3 to 11 carbon atoms and optionally being unsaturated with 1 or 2 unsaturated bonds,
the term heteroaryl denotes a monocyclic or bicyclic group wherein at least one of the rings is aromatic, containing from 5 to 11 ring members and containing from 1 to 4 hetero atoms selected from nitrogen, oxygen and sulphur,
the term heterocycloalkyl denotes a mono- or bi-cyclic group which is saturated or unsaturated with 1 or 2 unsaturated bonds, containing from 4 to 11 ring members and containing from 1 to 4 hetero atoms selected from nitrogen, oxygen and sulphur,
the expression “optionally substituted” when used in relation to aryl or arylalkyl, cycloalkyl or cycloalkylalkyl, heteroaryl or heteroarylalkyl, and heterocycloalkyl or heterocycloalkylalkyl groups means that the respective aryl, cycloalkyl, heteroaryl and heterocycloalkyl groups may be substituted by from 1 to 3 identical or different substituents selected from a halogen atom and the groups alkyl, alkoxy, alkylthio, alkylsulphinyl, alkylsulphonyl, hydroxy, mercapto, cyano, nitro, amino (optionally substituted by one or two alkyl groups), acyl, formyl, aminocarbonyl (optionally substituted on the nitrogen atom by one or two alkyl groups), acylamino (optionally substituted on the nitrogen atom by an alkyl group), alkoxycarbonyl, carboxy and sulpho,
the expression “optionally substituted” when used in relation to the groups pyrrolyl, piperidyl or piperazinyl means that the groups concerned may be substituted by from 1 to 3 identical or different groups selected from alkyl, alkoxy, aryl, arylalkyl, aryloxy and aryloxyalkyl.

An advantageous aspect of the invention relates to compounds of formula (I) wherein Alk represents an ethyl group.

Another advantageous aspect of the invention relates to compounds of formula (I) wherein R₈₀ and R₈₁ together form an oxo group, or wherein R₉₀ and R₉₁ together form an oxo group, or wherein R₈₀ and R₈₁ and also R₉₀ and R₉₁ form two oxo groups. More advantageously, R₈₀ and R₈₁ together form an oxo group and R₉₀ and R₉₁ each represent a hydrogen atom.

Preferred compounds of formula (I) are those wherein R₅ represents a hydrogen atom.

Other preferred compounds of formula (I) are those wherein R₃ and R₄ together form a methylenedioxy or ethylenedioxy (preferably methylenedioxy) group.

Advantageous compounds of formula (I) are those wherein R₂ represents a hydrogen atom.

An especially advantageous aspect of the invention relates to compounds of formula (I) wherein R₁ represents an optionally substituted aryl or optionally substituted arylalkyl group (preferably optionally substituted phenyl).

Another preferred aspect relates to compounds of the invention wherein G represents a hydroxy group.

Another likewise advantageous aspect relates to compounds of the invention wherein G represents *—X—C(X′)-Alk′-G′ wherein G′ represents a hydrogen atom.

Another advantageous aspect of the invention relates to compounds of formula (I) wherein G represents a group *—X—C(X′)-Alk′-NR₆R₇ wherein R₆ and R₇ form together with the nitrogen atom a 5- to 8-membered (more advantageously 6-membered), monocyclic (advantageously saturated) heterocycloalkyl group:

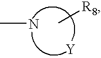

wherein Y represents a nitrogen atom, an oxygen atom or a CH₂ group (more advantageously CH₂) and R₈ represents a hydrogen atom or an alkyl group (more advantageously hydrogen).

Other preferred compounds are those belonging to the general formula (I) wherein Alk′ represents an alkylene group (more advantageously —CH₂—CH₂—).

Other preferred compounds of the invention are those wherein X and X′, which are the same or different, represent an oxygen atom or a sulphur atom (more advantageously oxygen).

An especially interesting compound of the invention is 7-ethyl-7-hydroxy-2,3-methylenedioxy-13-(4-methylphenyl)-9,12-dihydro-7H-cyclopenta[6,7]indolizino[1,2-b]-quinoline-8,10-dione.

The present invention relates also to a process for the preparation of compounds of formula (I), which process is characterised in that there is used as starting material a compound of formula (II) synthesised as described in EP 1 101 765:

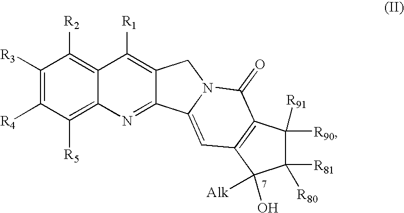

wherein Alk, R₁, R₂, R₃, R₄, R₅, R₈₀, R₈₁, R₉₀ and R₉₁ are as defined for formula (I),
   wherein the hydroxy group at C₇ is converted into X″H wherein X″ represents an SH, amino or alkylamino group to yield the compound of formula (III)

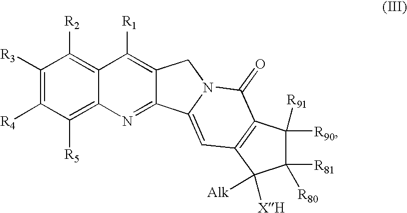

wherein Alk, R₁, R₂, R₃, R₄, R₅, R₈₀, R₈₁, R₉₀ and R₉₁ are as defined for formula (I) and X″ is as defined hereinbefore,
   which compounds of formula (II) or (III) are condensed with the reagent (IV):

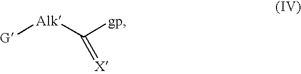

wherein G′, Alk′ and X′ are as defined for formula (I) and gp is a leaving group such as Hal, OH, SH, NR′R″ or OC(O)R′ wherein R′ and R″ represent alkyl groups,
   to yield the compound of formula (I),
   it being understood, for the purpose of simplifying the above process, that the reactive groups present in R₈₀, R₈₁, R₉₀ and R₉₁ may be protected by conventional protecting groups and deprotected at the appropriate point in time, that the hydroxy groups present in those same positions may be oxidised to oxo groups by conventional chemistry methods, and, conversely, the oxo groups present in those same positions may be reduced by conventional reducing agents at any appropriate point in time during synthesis, and that, when two of those groups together form a bond, the latter can be introduced at any point in time deemed useful by the person skilled in the art in order to facilitate synthesis,
   which compounds of formula (I):
   may be purified, if necessary, according to a conventional purification technique,
   are separated, where appropriate, into their stereoisomers according to a conventional separation technique,
   are converted, if desired, into addition salts thereof with a pharmaceutically acceptable acid or base.

Among the pharmaceutical compositions according to the invention, there may be mentioned more especially those that are suitable for oral, parenteral or nasal administration, tablets or dragées, sublingual tablets, capsules, lozenges, suppositories, creams, ointments, dermal gels etc.

The useful dosage varies according to the age and weight of the patient, the nature and severity of the disorder and the route of administration, which may be oral, nasal, rectal or parenteral (especially intravenous). The unit dose generally ranges from 0.1 to 500 mg per 24 hours for treatment in from 1 to 3 administrations.

The following Examples illustrate the invention but do not limit it in any way.

The structures of the compounds described in the Examples and the Preparations were determined according to the usual spectrophotometric techniques (infrared, NMR, mass spectrometry etc.).

The starting compounds of formulae (II) and (III′) wherein X represents an oxygen atom were synthesised under test conditions described in the patent specification EP 1 101 765 and adapted to the compounds of the invention using prior art documents known to the skilled person.

### EXAMPLE 1

### 7-Ethyl-7-hydroxy-2,3-methylenedioxy-13-(4-methylphenyl)-9,12-dihydro-7H-cyclopenta[6,7]indolizino[1,2-b]quinoline-8,10-dione

The title compound is prepared according to the method described in Example 11 of the patent specification EP 1 101 765, replacing the 2-bromo-3-bromomethyl-6,7-methylenedioxyquinoline by 2-bromo-3-bromomethyl-4-(4-methylphenyl)-6,7-methylenedioxyquinoline.

### Elemental Microanalysis

| | C % | H % | N % |
|---|---|---|---|
| Calculated: | 72.09 | 4.75 | 6.01 |
| Found: | 71.33 | 4.34 | 6.04 |

### EXAMPLE 2

### 7-Ethyl-7-hydroxy-2,3-methylenedioxy-13-(4-methoxyphenyl)-9,12-dihydro-7H-cyclopenta[6,7]indolizino[1,2-b]quinoline-8,10-dione

The title compound is prepared according to the method described in Example 1, replacing the 2-bromo-3-bromomethyl-4-(4-methylphenyl)-6,7-methylenedioxyquinoline by 2-bromo-3-bromomethyl-4-(4-methoxyphenyl)-6,7-methylenedioxyquinoline.

### EXAMPLE 3

### 7-Ethyl-2,3-methylenedioxy-13-(4-methylphenyl)-8,10-dioxo-8,9,10,12-tetrahydro-7H-cyclopenta[6,7]indolizino[1,2-b]quinolin-7-yl 3-piperidinopropanoate

To a suspension of 2 mmol of the compound of Example 1 in 150 ml of dichloromethane there are added, in succession, 1.13 g (7.2 mmol) of 3-piperidinopropanoic acid, 2.28 g (12.7 mmol) of 1-(3-dimethylaminopropyl)-3-ethylcarbodiimide hydrochloride and 0.34 g (2.78 mmol) of 4-dimethylaminopyridine. The reaction mixture is stirred for 24 hours at ambient temperature and then filtered. The filtrate is washed with sodium bicarbonate solution and then with water and is dried over magnesium sulphate. After concentrating the solvent in vacuo, the residue is dissolved in a solution of dichloromethane containing 30% ethanol. 0.57 ml of 1N hydrochloric acid is added and the precipitate formed is filtered off and recrystallised from acetonitrile to yield the expected compound.

### EXAMPLE 4

### 7-Ethyl-2,3-methylenedioxy-13-(4-methoxyphenyl)-8,10-dioxo-8,9,10,12-tetrahydro-7H-cyclopenta[6,7]indolizino[1,2-b]quinolin-7-yl 3-hexahydrocyclopenta[c]pyrrol-2(1H)-ylpropanoate

The title compound is prepared according to the method described in Example 3, starting from the compound of Example 2 and replacing the 3-piperidinopropanoic acid by 3-hexahydrocyclopenta[c]pyrrol-2(1H)-ylpropanoic acid.

### EXAMPLE 5

### 7-Ethyl-2,3-difluoromethylenedioxy-7-hydroxy-13-(4-methylphenyl)-9,12-dihydro-7H-cyclopenta[6,7]indolizino[1,2-b]quinoline-8,10-dione

The title compound is prepared according to the method described in Example 11 of the patent specification EP 1 101 765, replacing the 2-bromo-3-bromomethyl-6,7-methylenedioxyquinoline by 2-bromo-3-bromomethyl-4-(4-methylphenyl)-6,7-difluoromethylenedioxyquinoline.

### EXAMPLE 6

### 7-Ethyl-2,3-methylenedioxy-7-hydroxy-13-[4-(dimethylamino)phenyl]-9,12-dihydro-7H-cyclopenta[6,7]indolizino[1,2-b]quinoline-8,10-dione

The title compound is prepared according to the method described in Example 11 of the patent specification EP 1 101 765, replacing the 2-bromo-3-bromomethyl-6,7-methylenedioxyquinoline by 2-bromo-3-bromomethyl-4-[4-(dimethylamino)phenyl]-6,7-methylenedioxyquinoline.

### EXAMPLE 7

### 7-Ethyl-7-hydroxy-2,3-methylenedioxy-13-phenyl-9,12-dihydro-7H-cyclopenta[6,7]indolizino[1,2-b]quinoline-8,10-dione

The title compound is prepared according to the method described in Example 11 of the patent specification EP 1 101 765, replacing the 2-bromo-3-bromomethyl-6,7-methylenedioxyquinoline by 2-bromo-3-bromomethyl-4-phenyl-6,7-methylenedioxyquinoline.

### Pharmacological Study

### Example A

In vitro Activity

The murine leukaemia L1210 and the human colon carcinomas HCT116 and HT29 were used in vitro. The cells are cultured in RPMI 1640 complete culture medium containing 10% foetal calf serum, 2 mM glutamine, 50 units/ml of penicillin, 50 μg/ml of streptomycin and 10 mM Hepes, pH=7.4. The cells are distributed on microplates and are exposed to the cytotoxic compounds for 4 doubling times, that is to say 48 hours (L1210) or 96 hours (HCT116 and HT29). The number of viable cells is then quantified by a calorimetric assay, the Microculture Tetrazolium Assay (J. Carmichael et al., Cancer Res.; 47, 936-942, (1987)). The results are expressed in terms of the IC₅₀ (the concentration of cytotoxic agent which inhibits proliferation of the treated cells by 50%).

The compounds of the invention appear to be powerful cytotoxic agents, the IC₅₀ values being substantially below 1 μM.

By way of example, the compound of Example 1 has an IC₅₀ value of 3.2 nM (HT29) and the compound of Example 6 has an IC₅₀ value of 4.7 nM (HT29) and 10.4 nM (L1210).

### Example B

Pharmaceutical Composition

Preparation formula for 1000 tablets each containing 10 mg of active ingredient:

| | |
|---|---|
| Compound of Example 1 | 10 g |
| Hydroxypropylcellulose | 2 g |
| Wheat starch | 10 g |
| Lactose | 100 g |
| Magnesium stearate | 3 g |
| Talc | 3 g |

1-16. (canceled)

17. A compound selected from those of formula (I): 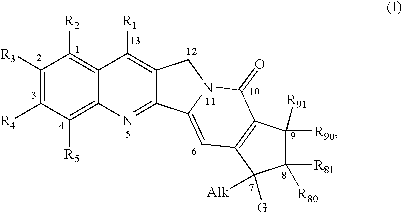
wherein:
Alk represents an alkyl group,
R₁, R₂, R₃, R₄ and R₅ are independently selected from a hydrogen atom, a halogen atom, an alkyl group, an alkenyl group, an alkynyl group, a polyhaloalkyl group, an optionally substituted cycloalkyl group, an optionally substituted cycloalkylalkyl group, a hydroxy group, a hydroxyalkyl group, an alkoxy group, an alkoxyalkyl group, a nitro group, a cyano group, an acyloxy group, a —C(O)—R group, and the groups —(CH₂)ₚ—NRₐR_{b}, —O—C(O)—NRₐR_{b} and —Z—Ar, wherein:
R represents an alkyl group, an alkoxy group or an amino group optionally substituted on the nitrogen atom by one or two alkyl groups;
p is an integer from 0 to 6;
Rₐ and R_{b} independently represent a hydrogen atom, an alkyl group, a cycloalkyl group, a cycloalkylalkyl group, an acyl group, an optionally substituted aryl group or an optionally substituted arylalkyl group, or Rₐ and R_{b} together with the nitrogen atom carrying them form a pyrrolyl, piperidyl or piperazinyl group, wherein each of these cyclic groups may be optionally substituted;
Z represents a bond, an oxygen atom, a sulphur atom or a group selected from —Z′—S(O)—, —S(O)ᵣ—Z′— and —Z′—(CR_{c}R_{d})_{q}—Z″—;
Ar represents an optionally substituted aryl or an optionally substituted heteroaryl group;
Z′ and Z″, which are the same or different, represent an oxygen atom, a sulphur atom, a group —NRₑ— or a bond;
R_{c}, R_{d} and Rₑ which are the same or different, represent a hydrogen atom or an alkyl group;
q represents an integer from 1 to 6;
and r represents an integer 1 or 2;
or two adjacent groups from R₂, R₃, R₄ and R₅ together with the carbon atoms carrying them form a group -T-(CR_{g}Rₕ)ₜ-T′-, wherein T and T′, which are the same or different, represent an oxygen atom, a sulphur atom or a group N—Rᵢ; R_{g} and Rₕ, which are the same or different, represent a hydrogen atom or a halogen atom; t is an integer from 1 to 3 inclusive; and Rᵢ represents a hydrogen atom, an alkyl group or a benzyl group,
R₈₀ and R₉₀ independently represent a hydrogen atom, a hydroxy group, an alkyl group or an alkoxy group,
R₈₁ and R₉₁ independently represent a hydrogen atom, an alkyl group, an alkenyl group or an alkynyl group, or, R₈₁ and R₉₁ together form a bond or an oxirane group, or two geminal groups, R₈₀ and R₈₁, and/or R₉₀ and R₉₁ together form an oxo group or a group —O—(CH₂)ₜ₁—O—, t₁ represents an integer from 1 to 3 inclusive,
G represents a group *—XH or *—X—C(X′)-Alk′-G′, wherein:
* represents the point of attachment to the C₇ carbon atom;
X and X′, which are the same or different, represent an oxygen atom, a sulphur atom, an amino group or an alkylamino group;
Alk′ represents an alkylene, alkenylene or alkynylene chain;
G′ represents a hydrogen atom or a group NR₆R₇ wherein:
i) R₆ and R₇ represent, each independently of the other, a hydrogen atom, an alkyl group, a cycloalkyl group, an optionally substituted aryl group, an optionally substituted arylalkyl group, an optionally substituted cycloalkyl group, an optionally substituted cycloalkylalkyl group, an optionally substituted heteroaryl group or an optionally substituted heteroarylalkyl group,
ii) or R₆ and R₇ together with the nitrogen atom carrying them form a 5- to 8-membered monocyclic heterocycloalkyl group 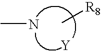
or a 5- to 11-membered bicyclic heterocycloalkyl group 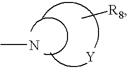
wherein:
Y represents a nitrogen atom, an oxygen atom or a CH₂ group and
R₈ represents a hydrogen atom, an alkyl group, an optionally substituted cycloalkyl group, an optionally substituted cycloalkylalkyl group, an optionally substituted aryl group, an optionally substituted arylalkyl group, an optionally substituted heterocycloalkyl group, an optionally substituted heterocycloalkylalkyl group, an optionally substituted heteroaryl group or an optionally substituted heteroarylalkyl group,
its enantiomers and diastereoisomers, and addition salts thereof with a pharmaceutically acceptable acid or base,
it being understood that at least one of the substituents R₁ to R₅ represents a group —Z—Ar,
and it being understood that:
alkyl means a linear or branched chain of from 1 to 6 carbon atoms,
alkenyl means a linear or branched chain of from 2 to 6 carbon atoms containing from 1 to 3 double bonds,
alkynyl means a linear or branched chain of from 2 to 6 carbon atoms containing from 1 to 3 triple bonds,
alkylene means a linear or branched divalent radical containing from 1 to 6 carbon atoms,
alkenylene means a linear or branched divalent radical containing from 2 to 6 carbon atoms and from 1 to 3 double bonds,
alkynylene means a linear or branched divalent radical containing from 2 to 6 carbon atoms and from 1 to 3 triple bonds,
acyl means a linear or branched alkyl-carbonyl radical containing from 1 to 6 carbon atoms,
alkoxy means an alkyl-oxy radical, the alkyl group of which is linear or branched and contains from 1 to 6 carbon atoms,
acyloxy means an acyl-oxy radical, the acyl group of which is a linear or branched alkylcarbonyl radical,
aryloxyalkyl means an aryl-oxy-alkyl group, the alkyl group of which is linear or branched and contains from 1 to 6 carbon atoms,
arylalkyl, cycloalkylalkyl, heteroarylalkyl and heterocycloalkylalkyl mean aryl-alkyl, cycloalkyl-alkyl, heteroaryl-alkyl and heterocycloalkyl-alkyl radicals, the alkyl group of which is a linear or branched chain of from 1 to 6 carbon atoms,
polyhaloalkyl means a linear or branched carbon chain containing from 1 to 3 carbon atoms and from 1 to 7 halogen atoms,
halogen means a fluorine atom, a chlorine atom, a bromine atom or an iodine atom,
aryl means a phenyl, naphthyl, indanyl, indenyl, dihydronaphthyl or tetrahydronaphthyl group,
cycloalkyl means a monocyclic or bicyclic hydrocarbon group containing from 3 to 11 carbon atoms and optionally being unsaturated with 1 or 2 unsaturated bonds,
heteroaryl means a monocyclic or bicyclic group wherein at least one of the rings is aromatic, containing from 5 to 11 ring members and containing from 1 to 4 hetero atoms selected from nitrogen, oxygen and sulphur,
heterocycloalkyl means a mono- or bi-cyclic group which is saturated or unsaturated with 1 or 2 unsaturated bonds, containing from 4 to 11 ring members and containing from 1 to 4 hetero atoms selected from nitrogen, oxygen and sulphur,
the expression “optionally substituted” when referring to aryl or arylalkyl, cycloalkyl or cycloalkylalkyl, heteroaryl or heteroarylalkyl, and heterocycloalkyl or heterocycloalkylalkyl groups means that these respective aryl, cycloalkyl, heteroaryl and heterocycloalkyl groups may be substituted by from 1 to 3 identical or different substituents selected from a halogen atom and the groups alkyl, alkoxy, alkylthio, alkylsulphinyl, alkylsulphonyl, hydroxy, mercapto, cyano, nitro, amino optionally substituted by one or two alkyl groups, acyl, formyl, aminocarbonyl optionally substituted on the nitrogen atom by one or two alkyl groups, acylamino optionally substituted on the nitrogen atom by an alkyl group, alkoxycarbonyl, carboxy and sulpho,
the expression “optionally substituted” when referring to the groups pyrrolyl, piperidyl or piperazinyl means that these groups may be substituted by from 1 to 3 identical or different groups selected from alkyl, alkoxy, aryl, arylalkyl, aryloxy and aryloxyalkyl.

18. The compound of claim 17, wherein Alk represents an ethyl group.

19. The compound of claim 17, wherein R₈₀ and R₈₁ together form an oxo group, or wherein R₉₀ and R₉₁ together form an oxo group, or wherein R₈₀ and R₈₁ and also R₉₀ and R₉₁ form two oxo groups.

20. The compound of claim 17, wherein R₅ represents a hydrogen atom.

21. The compound of claim 17, wherein R₃ and R₄ together form a methylenedioxy or ethylenedioxy group.

22. The compound of claim 17, wherein R₂ represents a hydrogen atom.

23. The compound of claim 17, wherein R₁ represents an optionally substituted aryl group or an optionally substituted arylalkyl group.

24. The compound of claim 17, wherein G represents a hydroxy group.

25. The compound of claim 17, wherein G represents *—X—C(X′)-Alk′-G′ wherein G′ represents a hydrogen atom.

26. The compound of claim 17, wherein G represents a group *—X—C(X′)-Alk′-NR₆R₇ wherein R₆ and R₇ form together with the nitrogen atom carrying them a 5- to 8-membered monocyclic heterocycloalkyl group 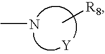
wherein Y represents a nitrogen atom, an oxygen atom or a group CH₂ and R₈ represents a hydrogen atom or an alkyl group.

27. The compound of claim 17, wherein Alk′ represents an alkylene group.

28. The compound of claim 17, wherein X and X′, which are the same or different, represent an oxygen atom or a sulphur atom.

29. The compound of claim 17, which is 7-ethyl-7-hydroxy-2,3-methylenedioxy-13-(4-methylphenyl)-9,12-dihydro-7H-cyclopenta[6,7]indolizino[1,2-b]quinoline-8,10-dione, its enantiomers, and addition salts thereof with a pharmaceutically acceptable acid or base.

30. A pharmaceutical composition comprising as active ingredient at least one compound of claim 17, alone or in combination with one or more inert, non-toxic, pharmaceutically acceptable excipients or carriers.

31. A method of treating a living animal body, including a human, afflicted with a cancer disease, comprising the step of administering to the living animal body, including a human, an amount of a compound of claim 17 which is effective for treatment of the disease.
